(19) United States
(12) Patent Application Publication
(10) Pub. No.: US 2007/0032754 A1
(43) Pub. Date: Feb. 8, 2007

(54) Method and apparatus for treating a wound

(76) Inventor: Richard F. Walsh, Fort Worth, TX (US)

Correspondence Address: BRACEWELL & GIULIANI LLP, P.O. BOX 61389, HOUSTON, TX, 77208-1389, US

(21) Appl. No.: 11/194,970

(22) Filed: Aug. 2, 2005

(51) Int. Cl. A61F 5/00

(52) U.S. Cl. 602/2

(57) ABSTRACT

A method for treating a wound is provided. The method includes applying a collagen material to the wound. The method also includes overlying a wound dressing material upon the collagen material on the wound. The method also includes draining fluids from the wound through a tube. The tube has a proximal end positioned adjacent the wound and a distal end coupled to a vacuum source. The fluids are drained from the wound by applying a negative pressure to the wound through the tube from the vacuum source.

### FIELD OF THE INVENTION

The present invention relates generally to a method and apparatus for treating a wound, and more particularly to applying a collagen material to a wound while applying negative pressure to the wound.

### BACKGROUND OF THE INVENTION

Wound dressings are typically applied over various types of wounds to promote healing and to reduce the risk of infection. Although various types of dressing materials have been successfully employed, wound dressings comprising semi-permeable materials are often preferred because they can increase patient comfort and lower the risk of infection. Semi-permeable wound dressings generally pass moisture vapors, but are generally impervious or impermeable to liquids. Thus, they can promote healing by permitting a wound site to breathe.

However, problems can arise with such wound dressings when they are placed over draining wounds because they tend to retain fluid. For example, surgical wounds often tend to drain for a post-operative period of about forty-eight hours. The fluid that can accumulate under such a semi-permeable wound dressing during a draining period can macerate the underlying tissue, cause infection and otherwise inhibit healing. A procedure for alleviating this problem involves periodically piercing the wound dressing, draining the accumulated fluids, and resealing the wound dressing opening. However, such a procedure is time-consuming for health care professionals and, unless it is conducted at relatively frequent intervals, can be relatively ineffective in dealing with the problems associated with trapped fluid accumulation. Other procedures which involve opening or changing wound dressings tend to have problems associated with exposing a wound to a greater risk of infection and can be uncomfortable for patients.

### SUMMARY

The present invention features a method for treating a wound. The method includes applying a collagen material to the wound. The method also includes overlying a wound dressing material upon the collagen material on the wound. The method also includes draining fluids from the wound through a tube. The tube has a proximal end positioned adjacent the wound and a distal end coupled to a vacuum source. The fluids are drained from the wound by applying a negative pressure to the wound through the tube from the vacuum source.

### BRIEF DESCRIPTION OF THE DRAWINGS

FIG. 1 shows a front view of the wound treatment device in accordance with the present invention.

FIG. 2 shows a top view of the wound treatment device of FIG. 1.

FIG. 3 shows a flow diagram illustrating an embodiment of a method in accordance with the present invention.

FIG. 4 shows a flow diagram illustrating a further embodiment of a method in accordance with the present invention.

### DETAILED DESCRIPTION OF THE INVENTION

Although the following detailed description contains many specific details for purposes of illustration, anyone of ordinary skill in the art will appreciate that many variations and alterations to the following details are within the scope of the invention. Accordingly, the exemplary embodiment of the invention described below is set forth without any loss of generality to, and without imposing limitations thereon, the claimed invention.

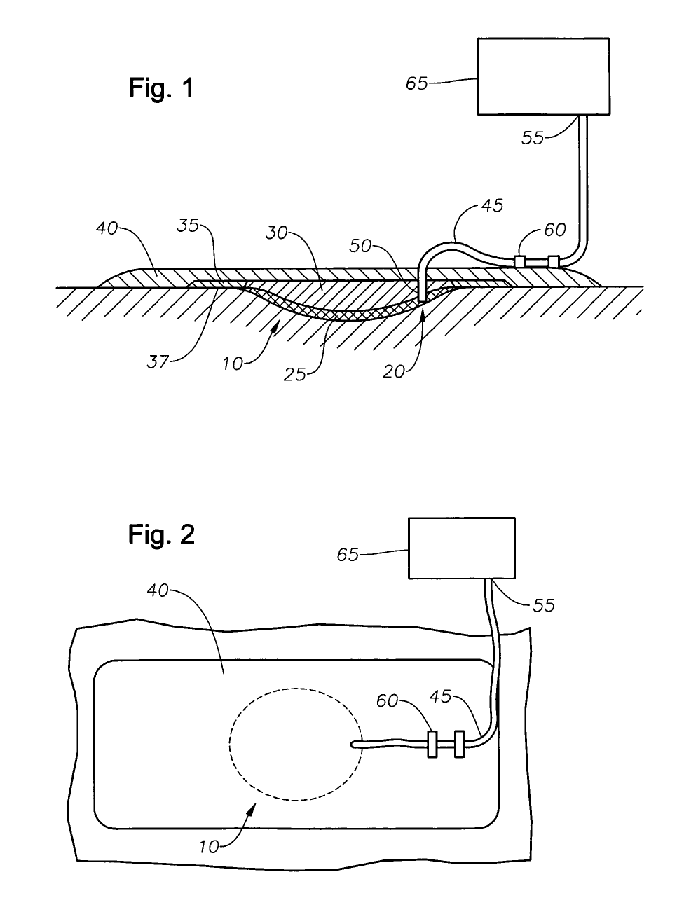

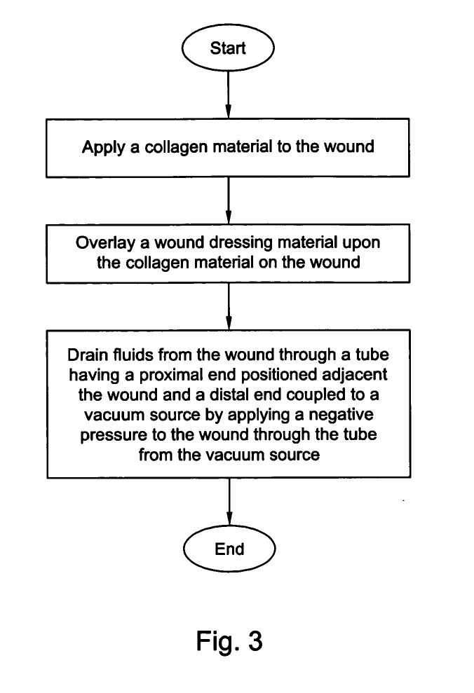

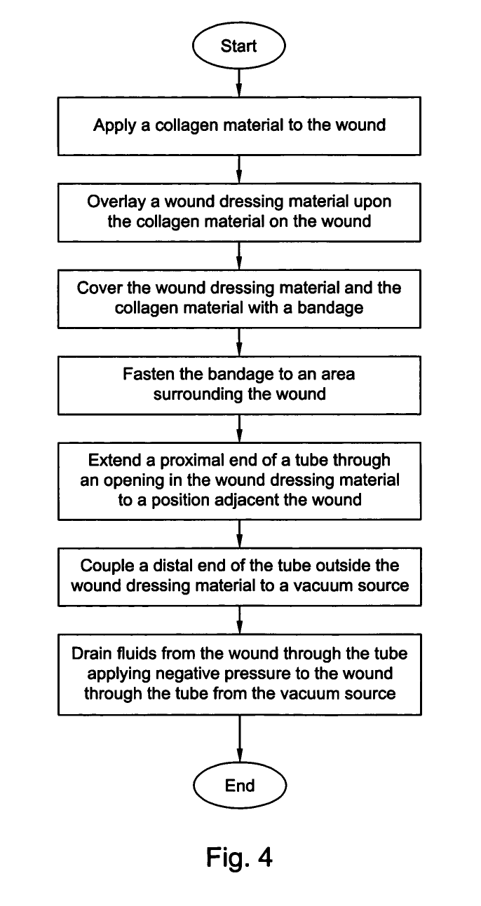

Referring to FIG. 1, a wound 10 is illustrated on a part of the body. The wound 10 can be, for example, a cut, scrape, scratch, sore, blister, incision, gouge, abrasion, or other type of wound. The first step in treating the wound 10 is to cleanse the wound 10 gently and carefully with a saline solution, a soap and water solution, a sterile water solution, or other non-toxic solution to the extent that this initial cleaning procedure is helpful to remove debris, necrotic slough, or other material on the wound 10. The water pressure during this cleansing typically should not exceed 8 psi. After the cleansing procedure, the wound 10 should be blotted dry by a sanitary towel or other material.

Thereafter, to treat the wound 10 in accordance with the method of the present invention, a collagen substance 25 is applied to the wound 10. Collagen 25 is a prevalent protein source that comes from various types of animals, including humans, as understood by those skilled in the art. Chains of amino acids make up collagen 25. Collagen 25 is a constituent of extracellular matrices and connective tissues from a vast variety of multicellular organisms. In these organisms, aggregates of collagen molecules coursing through the tissues are responsible for establishing and maintaining the physical integrity of diverse extracellular structures, thereby contributing to the functional capabilities of the organism as a whole. At the molecular level, collagen 25 is a protein containing lengthy domains of triple-helical conformation, as understood by those skilled in the art. The unique collagen fold is made possible by virtue of the repetitive Gly-X—Y sequences in participating chains. In this type of sequence, glycine occurs in every third position along the chain. Additionally, the collagen protein participates in the formation of extracellular aggregates that function primarily as supporting elements. Collagen 25 generally includes segments of triple-helical conformation and possesses the capacity for self-assembly into extracellular aggregates. As understood by those skilled in the art, such information on collagen structure and function has been derived largely from studies on selected higher vertebrate species, including humans.

The major physiological functions of collagen 25 are accomplished by extracellular aggregates of the molecules, and the structure of the aggregates is directly related to specific function. Several unique modes of aggregation have been discerned. For example, one of the most prevalent type of aggregate is the fiber, and the capacity to form fibers is common to many types of collagens. In general, fiber formation involves lateral association and axial displacement of molecules arranged in parallel. Bundled fibers lead to body tissue formation. Other known modes of aggregation involve associations of molecules allowing some degree of antiparallel orientation of individual molecules.

Collagen 25 is a natural biomaterial and is understood to be particularly useful for wound healing. Collagen 25 plays an integral part during each phase of wound healing and is an excellent hemostatic agent. It absorbs 40 to 60 times its weight in fluid. The most abundant and well characterized collagen 25 is type 1 extracted from bovine (cow) hide. Other sources include porcine (pig), chicken tendon, bovine tendon, and various others. The most preferential types of collagen 25 used in accordance with the method of the present invention are porcine collagen and bovine collagen. Also, as understood by those skilled in the art, the collagenous scaffold of the extracellular matrix includes at least 13 genetically distinct types of collagen, and up to 20 genetically distinct types of collagen, each of which can alternatively be used in accordance with the method of the present invention. Collagen types 1, 3, and 5 are typically specific for skin. The comprehensive reviews of the role of collagenous matrix in organ-specific tissue repair, as understood by those skilled in the art, illustrates the advantageous role collagens play in wound healing and tissue repair.

Collagen 25 is typically applied in a granular or particulate form, but can also be applied in other forms. The collagen particles 25 are applied as a relatively thin layer to the wound bed surface 10, such as for example a layer of collagen particles about 1/16 inch thick, that fully covers the wound bed surface 10. The collagen particles 25 are not layered thickly and are not packed tightly onto the wound bed surface 10. Thicker layers can be applied, however, to treat more severe wounds that feature heavy drainage and infection, such as for example a layer of collagen particles about ⅛ inch thick. Then, after drainage has slowed or the infection diminishes, a relatively thin layer of collagen particles 25 is applied to the wound bed surface 10. Therefore, more collagen particulate 25 is applied to heavily draining wounds, and less collagen particulate 25 is applied to lightly draining wounds.

If a topical medication is ordered for the wound 10, the topical medication is first applied to the wound 10 bed surface, and then collagen particles 25 are applied in a relatively thicker layer that completely covers the topical medication and wound bed surface 10. A further alternative of collagen use is utilizing a slurry that is freeze dried to attract moisture. After about 36 hours, the slurry forms a cake which can be grinded and applied to the wound 10.

After the collagen substance 25 is applied to the wound 10, a wound dressing material 30 is positioned overlying the collagen substance 25 on the wound 10. The wound dressing material 30 can be a semi-permeable or porous gauze that allows an amount of air to flow therethrough in order to let the wound 10 breathe. The wound dressing material 30 can also be in the form of a semi-permeable wound dressing that becomes impregnated with collagen 25 when applied overlying the collagen 25. Semi-permeable wound dressing materials 30 are often preferred because they can increase patient comfort and lower the risk of infection. Semi-permeable wound dressings generally allow moisture vapors to pass therethrough, but are generally impervious or impermeable to liquids. The wound dressing material 30 can also be an anti-microbial material to prevent infection and advance the healing of the wound 10.

After the wound dressing material 30 is placed upon the collagen substance 25 on the wound 10, a protective cream or ointment 35 can be applied on a peri-wound margin 37 of the wound 10 adjacent the collagen substance 25 and wound dressing material 30 to thereby prevent infection and provide additional protection and advance healing of the wound 10. A peri-wound margin 37 of the wound 10 is the area immediately surrounding the wound 10 that extends, for example, from the wound edge to about one inch or more from the wound edge. The amount a peri-wound margin 37 of the wound 10 extends from the wound edge often depends upon the size and depth of the wound 10. Many of the wound healing processes originate from the wound edges, which is called epithelization. The protective cream or ointment 35 protects a peri-wound margin 37 of the wound 10 and the wound edges from damage and maceration, enabling the epithelization to proceed properly.

After the collagen substance 25, wound dressing material 30, and protective cream 35 are applied to the wound 10, a bandage or drape 40 covers the collagen substance 25, wound dressing material 30, and protective cream 35. The bandage 40 can cover the entire surface area of the wound 10 and seal all treatment elements therein. The bandage 40 is fastened to an area surrounding the wound 10 to secure the collagen substance 25, wound dressing material 30, and protective cream 35 in a substantially fixed position.

A drain tube 45 is utilized to drain fluids 20 from the wound 10 to augment the healing process. The drain tube 45 has a proximal end 50 that extends into close proximity with the wound 10, and a distal end 55 that extends outward from the wound 10. The wound dressing material 30 and the cover bandage 40 can include an aperture or opening that facilitates insertion of the drain tube 45 therethrough. The proximal end 50 of the drain tube 45 is inserted through the aperture or opening in the wound dressing material 30 to a position adjacent the wound 10 to locate the proximal end 50 of the drain tube 45 in proper position to drain fluids 20 from the wound 10.

The distal end 55 of the drain tube 45 extends outward from the wound dressing material 30 and cover bandage 40 to a vacuum source 65, such as for example a vacuum pump or suction pump. The vacuum pump can be, for example, the Blue Sky Pump or the Medela Dominant 35 c/i Secretion and Suction Aspirator, both of which are commercially available from MEDELA AG. Such vacuum pumps can be used for a variety of suctioning procedures in addition to wound drainage of fluids 20, such as for example nasopharyngeal, tracheal, surgical, gastrointestinal, and thoracic drainage, as understood by those skilled in the art. Portions of the drain tube 45 that extend outward from the wound dressing material 30 and cover bandage 40 can be affixed or fastened to the cover bandage 40 by a fastener 60 to stabilize the proximal end 50 of the drain tube 45 in a substantially fixed position adjacent the wound 10. After portions of the drain tube 45 are affixed to the cover bandage 40, the distal end 55 of the drain tube 45 is coupled to the vacuum source 65.

After the proximal end 50 of the drain tube 45 is positioned in close proximity to the wound 10 adjacent the wound 10, the vacuum source 65 is activated. The vacuum source 65 applies a negative pressure or vacuum suction to the wound 10 through the drain tube 45, thereby draining fluids 20 from the wound 10 through the drain tube 45 and into the vacuum source 65. Drainage of fluids 20 from the wound 10 is an important part of the healing process, and it is beneficial to the healing process when such drainage fluids 20 from the wound 10 are removed from the wound area. Embodiments of the invention can provide suction of many wound drainage fluids 20 under various circumstances, such as surgical fluids, bodily fluids, gases, tissue, infectious materials from wounds, viscous puss-like fluids including white blood cells, cellular debris, and necrotic tissue, and other drained fluids, as understood by those skilled in the art. The negative pressure or vacuum suction is high enough to facilitate flow of wound drainage fluids 20 through the drain tube 45, but not so high that the negative pressure or vacuum suction disrupts the collagen formation 25 or destroys the healing structure of the wound itself.

The present invention offers important advantages and benefits. Application of the invention complies with and exceeds all applicable standards of care for medical or health care professionals, and complies with and exceeds all applicable regulatory requirements. The invention advantageously combines the beneficial use of collagen 25 with the draining of removable fluids 20 from the wound 10, while allowing the body's natural wound healing processes function properly, which are the complex sequential cellular activities of the wound 10 healing process necessary for wound closure. The invention advantageously establishes and maintains a moist, clean, and biologically active wound environment to sustain the sequential cellular activities. The invention also manages and controls the wound bed interference and such properties as improper moisture, inadequate oxygen, infection from bacteria, and other factors that affect the wound healing process. Application of the invention advantageously helps to reduce infection in chronic, acute, traumatic, subacute, and dehisced wounds, diabetic ulcers, pressure ulcers, flaps, grafts, and partial burns.

Although some embodiments of the present invention have been described in detail, it should be understood that various changes, substitutions, and alterations can be made hereupon without departing from the principle and scope of the invention. Accordingly, the scope of the present invention should be determined by the following claims and their appropriate legal equivalents.

That claimed is:

1. A method for treating a wound, comprising:
(a) applying a collagen material to the wound;
(b) overlying a wound dressing material upon the collagen material on the wound; and
(c) draining fluids from the wound through a tube having a proximal end positioned adjacent the wound and a distal end coupled to a vacuum source by applying a negative pressure to the wound through the tube from the vacuum source.

2. The method according to claim 1, wherein step (b) further comprises fastening a bandage to an area surrounding the wound to secure the wound dressing material and collagen material in a substantially fixed position.

3. The method according to claim 2, further comprising affixing the tube to the bandage to stabilize the proximal end of the tube in a substantially fixed position adjacent the wound.

4. The method according to claim 1, wherein the wound dressing material comprises a semi-permeable anti-microbial gauze material.

5. The method according to claim 1, wherein step (c) further comprises extending the proximal end of the tube through an aperture in the wound dressing material to a position adjacent the wound before draining fluids from the wound.

6. The method according to claim 1, further comprising applying a protective cream on a peri-wound margin of the wound adjacent the collagen material and wound dressing material.

7. The method according to claim 1, wherein the collagen material comprises a material selected from the group consisting of: porcine collagen, and bovine collagen.

8. The method according to claim 1, wherein step (a) comprises applying a topical medication to the wound, and thereafter applying a collagen material upon the topical medication on the wound.

9. A method for dressing a wound, comprising:
(a) applying a collagen material to the wound;
(b) overlying a wound dressing material upon the collagen material on the wound;
(c) covering the wound dressing material and the collagen material with a bandage;
(d) fastening the bandage to an area surrounding the wound;
(e) extending a proximal end of a tube through an opening in the wound dressing material to a position adjacent the wound;
(f) coupling a distal end of the tube outside the wound dressing material to a vacuum source; and
(g) draining fluids from the wound through the tube by applying negative pressure to the wound through the tube from the vacuum source.

10. The method according to claim 9, wherein the wound dressing material comprises a semi-permeable anti-microbial gauze material.

11. The method according to claim 9, further comprising applying a protective cream on a peri-wound margin of the wound adjacent the collagen material and wound dressing material.

12. The method according to claim 9, wherein the collagen material comprises a material selected from the group consisting of: porcine collagen, and bovine collagen.

13. The method according to claim 9, wherein step (a) comprises applying a topical medication to the wound, and thereafter applying a collagen material upon the topical medication on the wound.

14. An apparatus for treating a wound, comprising:
a collagen material for application on a wound;
a wound dressing material for overlying the collagen material on the wound;
a tube having a proximal end for extending adjacent the wound and a distal end for extending outwardly from the wound dressing material; and
a vaccum source coupled to the distal end of the tube and adapted to apply negative pressure to the wound, wherein the tube is adapted to be in fluid communication with the wound and the vacuum source.

15. The apparatus according to claim 14, further comprising a bandage for covering the wound dressing material and the collagen material, wherein the bandage is adapted to be fastened to an area surrounding the wound to secure the wound dressing material and collagen material in a substantially fixed position.

16. The apparatus according to claim 15, further comprising a fastener for affixing the tube to the bandage to stabilize the proximal end of the tube in a substantially fixed position adjacent the wound.

17. The apparatus according to claim 14, wherein the wound dressing material comprises a semi-permeable anti-microbial gauze material.

18. The apparatus according to claim 14, wherein the wound dressing material includes an aperture through which the proximal end of the tube extends.

19. The apparatus according to claim 14, further comprising a protective cream for application on a peri-wound margin of the wound adjacent the collagen material and wound dressing material.

20. The apparatus according to claim 14, wherein the collagen material comprises a material selected from the group consisting of: porcine collagen, and bovine collagen.

21. The apparatus according to claim 14, further comprising a topical medication for application to the wound, wherein the collagen material is for application upon the topical medication and the wound dressing material is for overlying the collagen material.
